(19) United States
(12) Patent Application Publication
(10) Pub. No.: US 2020/0357482 A1
(43) Pub. Date: Nov. 12, 2020

(54) METHOD AND SYSTEM FOR AUTOMATING CURATION OF GENETIC DATA

(71) Applicant: MedWhatBio, Inc., Spring Hill, FL (US)

(72) Inventor: Arturo DEVESA, Spring Hill, FL (US)

(73) Assignee: MedWhatBio, Inc., Spring Hill, FL (US)

(21) Appl. No.: 16/404,618

(22) Filed: May 6, 2019

(51) Int. Cl. G16B 30/00; G16B 20/00; G06N 20/00; G16B 25/00; G16B 50/00

(52) U.S. Cl. CPC G16B 30/00; G16B 20/00; G16H 50/20; G16B 25/00; G16B 50/00; G06N 20/00

(57) ABSTRACT

The present disclosure provides a method and system for automatic curation of genetic data. The system extracts text data from medical data received from corpus of medical database. In addition, the system creates word embedding of words present in the text data. Further, the system identifies variance explanation from the text data related to DNA variances. Furthermore, the system creates a user profile based on user genetic data and user data. Also, the system maps the user DNA variance from the user profile with the DNA variances to identify one or more characteristics. Also, the system generates a medical report based on the one or more characteristics.

### TECHNICAL FIELD

The present disclosure relates to the field of medical informatics, and in particular, relates to a method and system for automating curation of genetic data.

### BACKGROUND

Curation of genetic data to identify genetic variance is performed manually by a curator. The curator reads medical literatures like scientific paper, journal and research report on DNA variances. The curator identifies DNA variances from the medical literatures. In addition, the curator manually performs analysis of medical literature related to identified the DNA variances using the spreadsheet, DNA sequencing, DNA pairing and the like. Further, the curator manually identifies significance of the DNA variances in terms of type of variant, mutation or genetic disease by running a bioinformatics pipeline. Furthermore, the curator also identifies the list of genes and the genetic variances.

### SUMMARY

In a first example, a computer-implemented method is provided to automate curation of genetic data. The computer-implemented method may include a first step to extract text data from the medical data. The computer-implemented method may include a second step to create word embedding of words present in the text data in a low dimensional vector space. In addition, the computer-implemented method may include a third step to apply a training dataset on the text data. Further, the computer-implemented method may include a fourth step to identify variance explanation from the text data related to the DNA variances. Furthermore, the computer-implemented method may include a fifth step to create a user profile based on user genetic data and user data. Moreover, the computer-implemented method may include a sixth step to map the user DNA variance from the user profile with the DNA variances. Also, the computer-implemented method may include a seventh step to generate a medical report based on the one or more characteristics. The medical data is received from the corpus of medical database. The extraction of the text data is performed by using one or more machine learning algorithm. The medical data is received in a plurality of input forms. The corpus of medical database is created from one or more medical databases. The word embedding of words is created using one or more methods. The word embedding of words extracts text from the medical data present in the corpus of medical database. The training dataset is associated with a predetermined DNA variance data. The training dataset is applied for training the machine to identify genetic data related to DNA variances and the DNA sequence from the text data. The training dataset is applied in order to train the data curation system to perform automatic curation of the medical data. The DNA variances and the variance explanations are identified by analysis of the text data using the one or more machine learning algorithm. The identification is done after applying training dataset on word embedding of the text data. The identification is done in real time. The user profile is stored in profile database, wherein the user profile is created in real time. The mapping is done to indentify one or more characteristics associated with the user using one or more machine learning algorithms wherein the mapping is done in real time. The medical report comprises a plurality of results to be displayed on the one or more communication devices.

In an embodiment of the present disclosure, the user genetic data may include the user DNA sequences and the genome sequences of the user, wherein the user genetic data is received from one or more input devices in real time.

In an embodiment of the present disclosure, the one or more machine learning algorithms may includes a decision tree algorithm and a random forest algorithm. In addition, the one or more machine learning algorithms may include prediction algorithms, deep learning algorithms and natural language processing algorithm.

In an embodiment of the present disclosure, the user data may include name, age, gender, blood group, present disease and disease history of the user. The user data is entered by the user or an operator using the one or more communication devices. The user data is received in real time.

In an embodiment of the present disclosure, the one or more characteristics may include the genetic data, observed variant, the genetic variance and diseases related to the genetic variance.

In an embodiment of the present disclosure, the method may include a step to apply a training dataset on the text data, wherein the training dataset is associated with a predetermined DNA variance data. The training dataset is applied for training the machine to identify genetic data related to DNA variances from the text data.

In an embodiment of the present disclosure, the one or more medical databases may include medical university database, medical published database, medical institution data, genome project data and research database.

In an embodiment of the present disclosure, the genetic data may include DNA sequences, gene fusion, unique samples of genes, genetic mutation, mutation distribution, genes data, tissue distribution protein-protein interactions, open chromatin data, synthetic lethality data and tissue distribution.

In an embodiment of the present disclosure, the method may include a step to receive the user genetic data of a user from the one or more input devices and the user data of the user from one or more communication devices.

In an embodiment of the present disclosure, the plurality of results may include name, age, gender, blood group, variance explanation, suggestions, user DNA sequence, medical advice, user DNA variances, disease cause and health risk advice.

In a second example, a computer system is provided. The computer system includes one or more processors, and a memory. The memory is coupled to the one or more processors. The memory stores instructions. The memory is executed by the one or more processors. The execution of the memory causes the one or more processors to perform a method to automate the curation of genetic data. The method may include a first step to extract text data from the medical data. The method may include a second step to create word embedding of words present in the text data in a low dimensional vector space. In addition, the method may include a third step to apply a training dataset on the text data. Further, the method may include a fourth step to identify variance explanation from the text data related to the DNA variances. Furthermore, the method may include a fifth step to create a user profile based on user genetic data and user data. Moreover, the method may include a sixth step to map the user DNA variance from the user profile with the DNA variances. Also, the method may include a seventh step to generate a medical report based on the one or more characteristics. The medical data is received from the corpus of medical database. The extraction of the text data is performed by using one or more machine learning algorithm. The medical data is received in a plurality of input forms. The corpus of medical database is created from one or more medical databases. The word embedding of words is created using one or more methods. The word embedding of words extracts text from the medical data present in the corpus of medical database. The training dataset is associated with a predetermined DNA variance data. The training dataset is applied for training the machine to identify genetic data related to DNA variances and the DNA sequence from the text data. The training dataset is applied in order to train the data curation system to perform automatic curation of the medical data. The DNA variances and the variance explanations are identified by analysis of the text data using the one or more machine learning algorithm. The identification is done after applying training dataset on word embedding of the text data. The identification is done in real time. The user profile is stored in profile database, wherein the user profile is created in real time. The mapping is done to identify one or more characteristics associated with the user using one or more machine learning algorithms wherein the mapping is done in real time. The medical report comprises a plurality of results to be displayed on the one or more communication devices.

In a third example, a computer-readable storage medium is provided. The computer-readable storage medium encodes computer executable instructions that, when executed by at least one processor, performs a method to automate the curation of genetic data. The method may include a first step to extract text data from the medical data. The method may include a second step to create word embedding of words present in the text data in a low dimensional vector space. In addition, the method may include a third step to apply a training dataset on the text data. Further, the method may include a fourth step to identify variance explanation from the text data related to the DNA variances. Furthermore, the method may include a fifth step to create a user profile based on user genetic data and user data. Moreover, the method may include a sixth step to map the user DNA variance from the user profile with the DNA variances. Also, the method may include a seventh step to generate a medical report based on the one or more characteristics. The medical data is received from the corpus of medical database. The extraction of the text data is performed by using one or more machine learning algorithm. The medical data is received in a plurality of input forms. The corpus of medical database is created from one or more medical databases. The word embedding of words is created using one or more methods. The word embedding of words extracts text from the medical data present in the corpus of medical database. The training dataset is associated with a predetermined DNA variance data. The training dataset is applied for training the machine to identify genetic data related to DNA variances and the DNA sequence from the text data. The training dataset is applied in order to train the data curation system to perform automatic curation of the medical data. The DNA variances and the variance explanations are identified by analysis of the text data using the one or more machine learning algorithm. The identification is done after applying training dataset on word embedding of the text data. The identification is done in real time. The user profile is stored in profile database, wherein the user profile is created in real time. The mapping is done to indentify one or more characteristics associated with the user using one or more machine learning algorithms wherein the mapping is done in real time. The medical report comprises a plurality of results to be displayed on the one or more communication devices.

### BRIEF DESCRIPTION OF THE FIGURES

Having thus described the disclosure in general terms, reference will now be made to the accompanying figures, wherein;

FIG. 1 illustrates an interactive computing environment for curation of genetic data, in accordance with various embodiments of the present disclosure;

FIG. 2 is a flowchart of a method for the curation of the genetic data, in accordance with various embodiments of the present disclosure; and

FIG. 3 illustrates the block diagram of a computing device, in accordance with various embodiments of the present disclosure.

It should be noted that the accompanying figures are intended to present illustrations of exemplary embodiments of the present disclosure. These figures are not intended to limit the scope of the present disclosure. It should also be noted that accompanying figures are not necessarily drawn to scale.

### DETAILED DESCRIPTION

In the following description, for purposes of explanation, numerous specific details are set forth in order to provide a thorough understanding of the present technology. It will be apparent, however, to one skilled in the art that the present technology can be practiced without these specific details. In other instances, structures and devices are shown in block diagram form only in order to avoid obscuring the present technology.

Reference in this specification to “one embodiment” or “an embodiment” means that a particular feature, structure, or characteristic described in connection with the embodiment is included in at least one embodiment of the present technology. The appearance of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

Moreover, although the following description contains many specifics for the purposes of illustration, anyone skilled in the art will appreciate that many variations and/or alterations to said details are within the scope of the present technology. Similarly, although many of the features of the present technology are described in terms of each other, or in conjunction with each other, one skilled in the art will appreciate that many of these features can be provided independently of other features. Accordingly, this description of the present technology is set forth without any loss of generality to, and without imposing limitations upon, the present technology.

It should be noted that the terms “first”, “second”, and the like, herein do not denote any order, ranking, quantity, or importance, but rather are used to distinguish one element from another. Further, the terms “a” and “an” herein do not denote a limitation of quantity, but rather denote the presence of at least one of the referenced item.

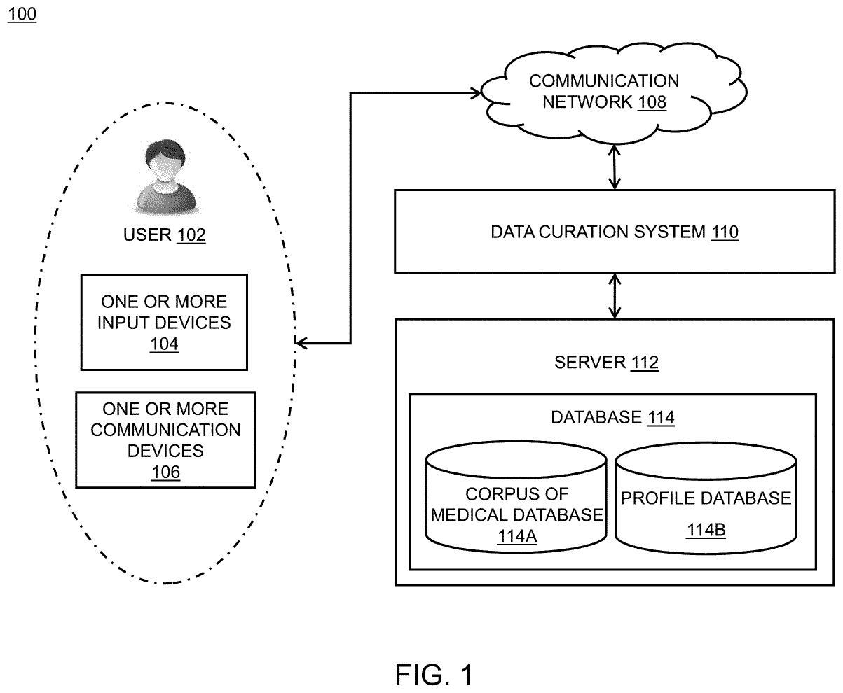

FIG. 1 illustrates an interactive computing environment 100 for automating process of curation of genetic data, in accordance with various embodiments of the present disclosure. The interactive computing environment 100 includes a user 102, one or more input devices 104 and one or more communication devices 106. In addition, the interactive computing environment 100 includes a communication network 108 and a data curation system 110. Further, the interactive computing environment 100 includes a server 112 and a database 114. The database 114 includes a corpus of medical database 114a and a profile database 114b. The above-stated components of the interactive computing environment 100 operate coherently and synchronously to enable curation of genetic data.

The interactive computing environment 100 includes the user 102. In an embodiment of the present disclosure, the user 102 is any person who wants medical assistance from a professional person having medical knowledge. In another embodiment of the present disclosure, the user 102 is any person who wants medical assistance from a medical practitioner. In another embodiment of the present disclosure, the user 102 is any person suffering from some disease. In another embodiment of the present disclosure, the user 102 wants to seek medical attention from the professional or the medical practitioner. In yet another embodiment of the present disclosure, the user 102 is any person who wants to know severity of the disease or sickness faced by the user 102. In yet embodiment of the present disclosure, the user 102 is a patient, an operator, lab technician and the like. In yet another embodiment of the present disclosure, the user 102 is a doctor, clinical geneticist, biomedical researcher, professor, and geneticist. In yet another embodiment of the present disclosure, the user 102 is any other person interested in the field of bioinformatics. The user 102 is associated with the one or more input devices 104 for sending and receiving information.

The interactive computing environment 100 includes the one or more input devices 104. The one or more input devices 104 includes but may not be limited to a video imaging device, an optical device, a color sensing device, and the like. The one or more input devices 104 receive or send a user genetic data. The user genetic data includes DNA sequence and genome sequence, and the like. In an embodiment of the present disclosure, the user genetic data include but may not be limited genes fusion, protein-protein interactions and phenotype information. In general, DNA sequence refers to determining the order of the four chemical building blocks called “bases” that make up the DNA molecule. The DNA sequence facilitates information related to the genes carried in a particular DNA segment. In an example, the DNA sequence is used to determine stretches containing genes and regulatory instructions. The stretches are used for turning genes on or off for performing specific functionality in a human body. In addition, DNA sequence can highlight changes in a gene that may cause disease in the human body. Also, the one or more input devices 104 provide the user genetic data to the data curation system 110.

The interactive computing environment 100 includes the one or more communication devices 106. The one or more communication devices 106 includes but may not be limited to a computer, smart television, electronic tablet, smartphone, gesture-controlled devices and the like. The one or more communication devices 106 receive or send information entered by the user 102 on the one or more communication devices 106. The user data is associated with the user 102. The user data includes but may not be limited to name, age, gender, weight, height, blood group, disease and illness history. The one or more communication devices 106 performs computing operations based on operating system installed inside the one or more communication devices 106. In general, the operating system is system software that manages computer hardware and software resources and provides common services for computer programs. In addition, the operating system acts as an interface for software installed inside the one or more communication devices 106 to interact with hardware components of the one or more communication devices 106.

In an embodiment of the present disclosure, the operating system installed inside the one or more communication devices 106 is a mobile operating system. In an embodiment of the present disclosure, the one or more communication devices 106 performs computing operations based on any suitable operating system designed for the one or more communication devices 106. In an example, the operating system includes Windows operating system, Android operating system, and Symbian operating system. In another example, the operating system includes Bada operating system, ios operating and BlackBerry operating system. In an embodiment of the present disclosure, the operating system is any other operating system suitable for performing computation and provide interface to the user on the one or more communication devices 106. In an embodiment of the present disclosure, the one or more communication devices 106 operates on any version of particular operating system of above mentioned operating systems.

In another embodiment of the present disclosure, the one or more communication devices 106 performs computing operations based on any suitable operating system designed for the one or more communication devices 106. In an example, the operating system installed inside the one or more communication devices 106 is Windows. In another example, the operating system installed inside the one or more communication devices 106 is Mac. In yet another example, the operating system installed inside the one or more communication devices 106 is Linux based operating system. In yet another example, the operating system installed inside the one or more communication devices 106 may be one of UNIX, Kali Linux, and the like. However, the operating system is not limited to above mentioned operating systems.

In an embodiment of the present disclosure, the one or more communication devices 106 operate on any version of Windows operating system. In another embodiment of the present disclosure, the one or more communication devices 106 operate on any version of Mac operating system. In another embodiment of the present disclosure, the one or more communication devices 106 operate on any version of Linux operating system. In yet another embodiment of the present disclosure, the one or more communication devices 106 operates on any version of particular operating system of the above mentioned operating systems. The one or more communication devices 106 are associated with the communication network 108 for transferring and receiving data.

The interactive computing environment 100 includes the communication network 108 which acts as a medium for transferring and receiving data. In an embodiment of the present disclosure, the communication network 108 facilitates in network connectivity between the one or more communication devices 106 and the data curation system 110. In another embodiment of the present disclosure, the communication network 108 facilitates in network connectivity between the one or more input devices 104 and the data curation system 110. In another embodiment of the present disclosure, the communication network 108 may be any type of network that provides internet connectivity to the data curation system 110. In yet embodiment of the present disclosure, the communication network 108 is a wireless mobile network. In yet embodiment of the present disclosure, the communication network 108 is a wired network with a finite bandwidth. In yet another embodiment of the present disclosure, the communication network 108 is combination of the wireless and the wired network for optimum throughput of data transmission. In yet another embodiment of the present disclosure, the communication network 108 is an optical fiber high bandwidth network that enables high data rate with negligible connection drops. In yet another embodiment of the present disclosure, the communication network 108 provides medium for the one or more communication devices 106 to connect to the data curation system 110.

The interactive computing environment 100 includes the data curation system 110. The data curation system 110 facilitates in automating the process of curation of the genetic data. In an embodiment of the present disclosure, the data curation system 110 is accessed through a web browser on the one or more communication devices 106. In another embodiment of the present disclosure, the data curation system 110 is accessed through a widget, API, web applets and the like. In an example, the web-browser includes but may not be limited to Opera, Mozilla Firefox, Google Chrome, Internet Explorer, Microsoft Edge, Safari and UC Browser. Further, the web browser runs on any version of the respective web browser of the above mentioned web browsers. The user 102 views the data curation system 110 on the one or more communication devices 106 through the communication network 108.

The data curation system 110 is associated with the server 112. In an embodiment of the present disclosure, the data curation system 110 is installed at the server 112. In another embodiment of the present disclosure, the data curation system 110 is installed at a plurality of servers. In general, the server 112 refers to a computer that provides data to other computers. It may serve data to systems on a local area network (LAN) or a wide area network (WAN) over the Internet. Many types of servers exist, including web servers, mail servers, file servers, application server and the like. Each type of server runs on a software specific to the purpose of the server 112. In an example, a Web server may run Apache HTTP Server or Microsoft IIS, which both provide access to websites over the Internet. A mail server may run a program like Exim or iMail, which provides SMTP services for sending and receiving email. A file server might use Samba or the operating system's built-in file sharing services to share files over a network. The plurality of servers communicates with each other using the communication network 108. In yet another embodiment of the present disclosure, the data curation system 110 is located in the server 112. In an embodiment of the present disclosure, the server 112 is a cloud server. In general, the cloud server possesses and exhibit similar capabilities and functionality to the server 112 but is accessed remotely from a cloud service provider. In an example, the server 112 is similar to a physical server but provides virtual space for handling all the operations.

In an embodiment of the present disclosure, the server 112 receives data from the database 114. In general, database refers to a data structure that stores information in an organized manner. The database 114 stores information in multiple tables, which may each include several different fields. In an example, a company database may include tables for products, employees, and financial records. Each of these tables would have different fields that are relevant to the information stored in the table. In an embodiment of the present disclosure, the database 114 is a cloud based database for storing information which is provided as service to the user 102 for accessing it using cloud computing platform. In another embodiment of the present disclosure, the database 114 is any other database based on the requirement of the data curation system 110.

The database 114 includes the corpus of medical database 114a and the profile database 114b. The corpus of medical database 114a is created based on one or more medical databases. The one or more medical databases include but may not be limited to medical university database and medical published database. The one or more medical databases include but may not be limited to medical institution data, genome project data and research databases. The corpus of medical database 114a is updated on periodic basis. In an embodiment of the present disclosure, the periodic basis include but may not be limited to weekly, monthly, daily, yearly, hourly and quarterly. The data curation system 110 receives data from the one or more databases in real time. In an embodiment of the present disclosure, the data curation system 110 integrates with the one or more medical databases for receiving medical data. The medical data received from the one or more databases is used for creating the corpus of medical database 114a. The medical data created in the corpus of medical database is in a plurality of input forms. The plurality of input forms includes but may not be limited to text, image, audio, video, gif, animation and the like. In addition, the one or more medical databases are magazine database, genome project database research database, and the like. The corpus of medical database 114a includes data in form of text, image, picture, literature, journal, audio, video and the like. The data present in the corpus of medical database 114a is associated with genetics of the human body. The profile database 114b includes the user profile of the user 102. The profile database 114b includes information related to the user 102.

The data curation system 110 extracts text data from the medical data received from the corpus of medical database 114a. The extraction of the text data is performed by using one or more machine learning algorithms. In an embodiment of the present disclosure, the one or more machine learning algorithms includes a decision tree algorithm and a random forest algorithm. In another embodiment of the present disclosure, the one or more machine learning algorithms include but may not be limited to prediction algorithms, deep learning algorithms, natural language processing algorithm and the like. However, the one or more machine learning algorithms are not limited to the above-mentioned algorithms.

The data curation system 110 creates word embedding of words present in the text data in a low dimensional vector space. The word embedding of words is created for the text data extracted from the medical data present in the corpus of medical database 114a. In general, the word embedding of the words is a learned representation for text where words that have the same meaning have similar representation. In an embodiment of the present disclosure, the data curation system 110 creates sentence embedding of sentence occurring in the text data. The word-embedding of words is created using one or more methods. The one or more methods used to create the word embedding includes recurrent neural networks, convolutional neural networks, word embedding layer, word2vec algorithm, glove algorithm and the like. In an embodiment of the present disclosure, the data curation system 110 uses recurrent neural networks to create the sentence embedding of sentences occurring in the text data. In another embodiment of the present disclosure, the data curation system 110 uses convolutional neural networks to create the sentence embedding of sentences occurring in the text data. However, the data curation system 110 is not limited to above mentioned networks and methods to create the sentence embedding of sentences occurring in the text data.

The data curation system 110 receives a training dataset of a predetermined DNA variance data. The training dataset facilitates the machine learning algorithms to learn curation of genetic data. The training dataset is created from one or more sources. The one or more sources include but may not be limited to medical literature, textbooks, online databases, journal articles, graphics, podcasts, videos, animations and medical data warehouses.

The data curation system 110 applies the training dataset on the text data. The training dataset is associated with a predetermined DNA variance data. The training dataset is applied for training the machine to identify genetic data related to DNA variances and the DNA sequence from the text data. The training dataset is received from a corpus of medical database 114a. In an embodiment of the present disclosure, the training is applied by using the one or more machine learning algorithm. In another embodiment of the present disclosure, the training is applied by using deep learning algorithm or artificial intelligence based algorithm for the automatic curation of the genetic data. In an embodiment of the present disclosure, the genetic data includes but may not be limited to DNA sequences, genes fusion, unique samples of genes and samples of genes with mutations. In an embodiment of the present disclosure, the genetic data includes mutation distribution, tissue distribution, protein-protein interactions, open chromatin data and synthetic lethality data. In an embodiment of the present disclosure, the genetic data includes gene expression profiles across various experimental conditions or phenotypes, open chromatin data, histone modification and the like. The training is applied in order to train the data curation system 110 to perform automatic curation of the genetic data from the medical data received from the corpus of medical database 114a. In an embodiment of the present disclosure, the unstructured data present in the training dataset is analyzed by using the one or more machine learning algorithm. In an embodiment of the present disclosure, semi-structured data present in the training dataset is analyzed by using the one or more machine learning algorithm. The analysis of the training dataset is performed in order to form structured data from the unstructured data of the training dataset.

In an embodiment of the present disclosure, the training dataset includes the predetermined DNA variance data. In an embodiment of the present disclosure, the predetermined DNA variance data does not include all the DNA variances. In another embodiment of the present disclosure, the predetermined DNA variance data includes all the DNA sequences and the DNA variances. The predetermined DNA variance data is extracted from the corpus of medical database 114a. In an embodiment of the present disclosure, the predetermined DNA variance data provides data and information about the DNA variances that may be required by the user 102 in future.

In an embodiment of the present disclosure, the training dataset includes the plurality of medical articles. The plurality of medical articles is extracted from the one or more sources. The plurality of medical articles provides medical facts outside the context of DNA sequences. The plurality of medical facts is used to train the data curation system 110 for curation of genetic data. In another embodiment of the present disclosure, the training dataset includes the plurality of DNA sequences extracted from the one or more sources.

The data curation system 110 performs analysis of the text data based on the training dataset. The analysis is performed by using the one or more machine learning algorithms. The analysis is performed in order to identify the DNA variances from the text data present in the plurality of input forms in the corpus of medical database 114a. The analysis is performed based on the training of the data curation system 110. The analysis is performed for the curation of the genetic data from the medical data received from the corpus of medical database 114a. In addition the data curation system 110 identifies variance explanation from the text data related to DNA variances in the text data. The DNA variances and the variance explanations are identified by analysis of the text data using the one or more machine learning algorithms. The identification is done after applying training dataset on word embedding of the text data.

In addition, the data curation system 110 receives the user genetic data from the one or more input devices 104. The data curation system 110 receives user data from the one or more communication devices 106. The user genetic data and the user data is received at the data curation system 110 with the assistance of the communication network 108. Further, the data curation system 110 creates the user profile of the user 102 based on the user genetic data and the user data. The user profile is stored in profile database 114b. The user profile includes but may not be limited to user DNA sequence, user genome sequence, name, age, gender, weight, height, blood group, illness history and disease. In an embodiment of the present disclosure, the user profile includes user DNA variances, user mutations, user genome, user etiological information and the like.

Furthermore, the data curation system 110 maps the user profile with the user DNA variance from the user profile with the DNA variances. The mapping is done to identify one or more characteristics associated with the user. The mapping is done by using one or more machine learning algorithms. The one or more characteristics includes the genetic data, observed variant as deleterious or tolerable, the genetic variance, diseases related to the genetic variance, and the like. In an embodiment, the data curation system 110 collectively receives data for mapping from the one or more input devices 104, the one or more communication devices 106, the corpus of medical database 114a and the profile database 114b. The mapping is done to identify the related disease and mutations based on the user DNA variance. The mapping facilitates identification of the meaning of the DNA variance for the user 102. In an example, the genetic data and the user DNA variance related to the user 102 is mapped with the DNA variance identified from the text data. The mapping shows a previously unreported variant PVT1 and GSTP1 which are likely to be pathogenic based on the variance explanation and needs medical advice. In another example, the data curation system 110 maps the phenotypic information of the user 102 with the causative genes for genetic disease and associated phenotypes resulting in phenotype associated with the genes. Moreover, the data curation system 110 generates medical report based on the one or more characteristics identified from the mapping of the user profile with the DNA variance from the text data. The medical report includes a plurality of results based on the one or more characteristics. The plurality of results include but may not be limited to name, age, gender, blood group, user DNA variance, variance explanation, suggestions, user DNA sequence and medical advice. In addition, the plurality of results include but may not be limited to drug advice, precautions, health risk advice, disease cause and personalized prescription. The medical report is present in any form such as pie charts, bar graphs, text, digital files, and the like. The medical report is displayed on the one or more communication devices 106 to the user 102. In an example, the report includes DNA variances, mutations, etiological information, drug advice, suggestions, precautions, health risk advice, personalized prescriptions, and the like.

In an embodiment, the data curation system 110 may be trained in any one of one or more languages. Further, the data curation system 110 may respond to the user 102 in specified language of the one or more languages. In an embodiment of the present disclosure, the data curation system 110 is enabled in English language. In another embodiment of the present disclosure, data curation system 110 is enabled in Hindi language. In yet another embodiment of the present disclosure, the data curation system 110 is enabled in any language of the one or more languages such as Spanish, French, German, Hindi, Chinese, Japanese, and the like.

The data curation system 110 performs automatic curation of data in order to reduce the time required in the manual curation of the genetic data by the curator. The data curation system 110 identifies the list of genes and the genetic variants used for the research purpose. The data curation system 110

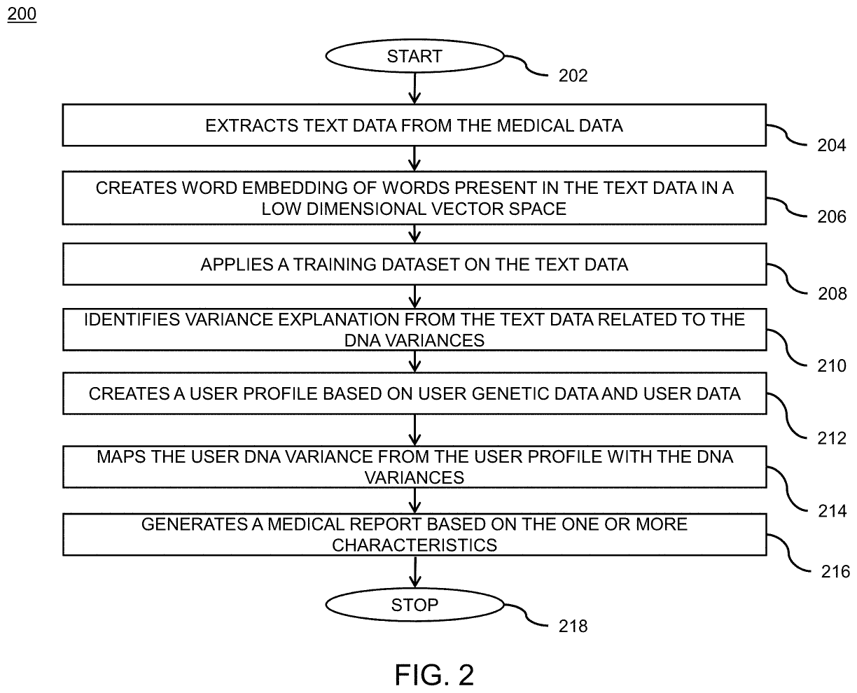

FIG. 2 is a flowchart 200 of a method for the curation of genetic data, in accordance with various embodiments of the present disclosure. The flowchart 200 initiates at step 202. Following step 202, at step 204 the data curation system 110 extracts text data from the medical data. At step 206, the data curation system 110 creates word embedding of words present in the text data in a low dimensional vector space. At step 208, the data curation system 110 applies a training dataset on the text data. At step 210, the data curation system 110 identifies variance explanation from the text data related to the DNA variances. At step 212, the data curation system 110 creates a user profile based on user genetic data and user data. At step 214, the data curation system 110 maps the user DNA variance from the user profile with the DNA variances. At step 216, the data curation system 110 generates a medical report based on the one or more characteristics. The flow chart 200 terminates at step 218.

It may be noted that the flowchart 200 is explained to have above stated process steps; however, those skilled in the art would appreciate that the flowchart 200 may have more/less number of process steps which may enable all the above-stated embodiments of the present disclosure.

In an embodiment, the data curation system 110 may be implemented using a single computing device, or a network of computing devices, including cloud-based computer implementations. The computing devices are preferably server class computers including one or more high-performance computer processors and random-access memory and running an operating system such as LINUX or variants thereof. The operations of the data curation system 110 as described herein can be controlled through either hardware or through computer programs installed in a non-transitory computer-readable storage devices such as solid-state drives or magnetic storage devices and executed by the processors to perform the functions described herein. The database 114 is implemented using non-transitory computer-readable storage devices, and suitable database management systems for data access and retrieval. The data curation system 110 includes other hardware elements necessary for the operations described herein, including network interfaces and protocols, input devices for data entry, and output devices for display, printing, or other presentations of data. Additionally, the operations listed here are necessarily performed at such a frequency and over such a large set of data that they must be performed by a computer in order to be performed in a commercially useful amount of time.

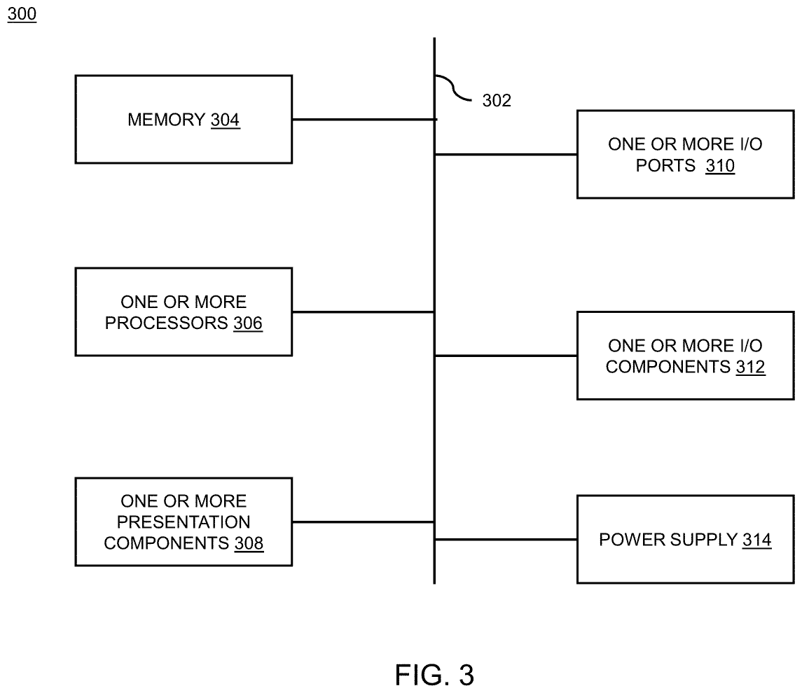

FIG. 3 illustrates a block diagram of the device 300, in accordance with various embodiments of the present disclosure. The device 300 includes a bus 302 that directly or indirectly couples the following devices: memory 304, one or more processors 306, one or more presentation components 308, one or more input/output (I/O) ports 310, one or more input/output components 312, and an illustrative power supply 314. The bus 302 represents what may be one or more busses (such as an address bus, data bus, or combination thereof). Although the various blocks of FIG. 3 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. The inventors recognize that such is the nature of the art, and reiterate that the diagram of FIG. 3 is merely illustrative of an exemplary device 300 that can be used in connection with one or more embodiments of the present invention. Distinction is not made between such categories as “workstation,” “server,” “laptop,” “hand-held device,” etc., as all are contemplated within the scope of FIG. 3 and reference to “computing device.”

The device 300 typically includes a variety of computer-readable media. The computer-readable media can be any available media that can be accessed by the device 300 and includes both volatile and nonvolatile media, removable and non-removable media. By way of example, and not limitation, the computer-readable media may comprise computer storage media and communication media. The computer storage media includes volatile and nonvolatile, removable and non-removable media implemented in any method or technology for storage of information such as computer-readable instructions, data structures, program modules or other data. The computer storage media includes, but is not limited to, non-transitory computer-readable storage medium that stores program code and/or data for short periods of time such as register memory, processor cache and random access memory (RAM), or any other medium which can be used to store the desired information and which can be accessed by the device 300. The computer storage media includes, but is not limited to, non-transitory computer readable storage medium that stores program code and/or data for longer periods of time, such as secondary or persistent long term storage, like read-only memory (ROM), EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the device 300. The communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 304 includes computer-storage media in the form of volatile and/or nonvolatile memory. The memory 304 may be removable, non-removable, or a combination thereof. Exemplary hardware devices include solid-state memory, hard drives, optical-disc drives, etc. The device 300 includes the one or more processors 306 that read data from various entities such as memory 304 or I/O components 312. The one or more presentation components 308 present data indications to the user 102 or other device. Exemplary presentation components include a display device, speaker, printing component, vibrating component, etc. The one or more I/O ports 310 allow the device 300 to be logically coupled to other devices including the one or more I/O components 312, some of which may be built in. Illustrative components include a microphone, joystick, game pad, satellite dish, scanner, printer, wireless device, etc.

The foregoing descriptions of pre-defined embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed:

1. A computer-implemented method for automating curation of genetic data, the computer-implemented method comprising:
extracting, at a data curation system with a processor, text data from medical data, wherein the medical data is received from a corpus of medical database, wherein the extraction of the text data is performed by using one or more machine learning algorithm, wherein the medical data is received in a plurality of input forms, wherein the corpus of medical database is created from one or more medical databases;
creating, at the data curation system with the processor, word embedding of words present in the text data in a low dimensional vector space, wherein the word embedding of words is created using one or more methods, wherein the word embedding of words extracts text from the medical data present in the corpus of medical database;
applying, at the data curation system with the processor, a training dataset on the text data, wherein the training dataset is associated with a predetermined DNA variance data, wherein the training dataset is applied for training a machine to identify genetic data related to DNA variances from the text data, the training dataset is applied in order to train the data curation system to perform automatic curation of the medical data;
identifying, at the data curation system with a processor, variance explanation from the text data related to the DNA variances, wherein the DNA variances and the variance explanation are identified by analysis of the text data using the one or more machine learning algorithm, wherein the identification is done after applying the training dataset on the word embedding of the text data, wherein the identification is done in real time;
creating, at the data curation system with the processor, a user profile based on user genetic data and user data, wherein the user profile is stored in profile database, wherein the user profile is created in real time;
mapping, at the data curation system with the processor, the user DNA variance from the user profile with the DNA variances, wherein the mapping is done to identify one or more characteristics associated with the user using one or more machine learning algorithms, wherein the mapping is done in real time; and
generating, at the data curation system with the processor, a medical report based on the one or more characteristics, wherein the medical report comprises a plurality of results to be displayed on one or more communication devices.

2. The computer-implemented method as recited in claim 1, wherein the user genetic data comprises the user DNA sequences and the genome sequences of the user, wherein the user genetic data is received from one or more input devices in real time.

3. The computer-implemented method as recited in claim 1, wherein the one or more machine learning algorithms includes a decision tree algorithm, a random forest algorithm, prediction algorithms, deep learning algorithms and natural language processing algorithm.

4. The computer-implemented method as recited in claim 1, wherein the user data comprises name, age, gender, blood group, present disease and disease history of the user, wherein the user data is entered by the user or an operator using the one or more communication devices, wherein the user data is received in real time.

5. The computer-implemented method as recited in claim 1, wherein the one or more characteristics comprises the genetic data, observed variant, the genetic variance and diseases related to the genetic variance.

6. The computer-implemented method as recited in claim 1, further comprising applying, at the data curation system with the processor, a training dataset on the text data, wherein the training dataset is associated with a predetermined DNA variance data, wherein the training dataset is applied for training the machine to identify genetic data related to DNA variances from the text data.

7. The computer-implemented method as recited in claim 1, wherein the one or more medical databases comprises medical university database, medical published database, medical institution data, genome project data and research database.

8. The computer-implemented method as recited in claim 1, wherein the genetic data comprises DNA sequences, gene fusion, unique samples of genes, genetic mutation, mutation distribution, genes data, tissue distribution protein-protein interactions, open chromatin data, synthetic lethality data and tissue distribution.

9. The computer-implemented method as recited in claim 1, further comprising receiving, at the data curation system with the processor, the user genetic data of a user from one or more input devices and the user data of the user from one or more communication devices.

10. The computer-implemented method as recited in claim 1, wherein the plurality of results comprises name, age, gender, blood group, variance explanation, suggestions, user DNA sequence, medical advice, user DNA variances, disease cause and health risk advice.

11. A computer system comprising:
one or more processors; and
a memory coupled to the one or more processors, the memory for storing instructions which, when executed by the one or more processors, cause the one or more processors to perform a method for automating curation of genetic data, the method comprising:
extracting, at a data curation system, text data from medical data, wherein the medical data is received from a corpus of medical database, wherein the extraction of the text data is performed by using one or more machine learning algorithm, wherein the medical data is received in a plurality of input forms, wherein the corpus of medical database is created from one or more medical databases;
creating, at the data curation system, word embedding of words present in the text data in a low dimensional vector space, wherein the word embedding of words is created using one or more methods, wherein the word embedding of words extracts text from the medical data present in the corpus of medical database;
applying, at the data curation system, a training dataset on the text data, wherein the training dataset is associated with a predetermined DNA variance data, wherein the training dataset is applied for training a machine to identify genetic data related to DNA variances from the text data, the training dataset is applied in order to train the data curation system to perform automatic curation of the medical data;
identifying, at the data curation system, variance explanation from the text data related to the DNA variances, wherein the DNA variances and the variance explanation are identified by analysis of the text data using the one or more machine learning algorithm, wherein the identification is done after applying the training dataset on the word embedding of the text data, wherein the identification is done in real time;
creating, at the data curation system, a user profile based on user genetic data and user data, wherein the user profile is stored in profile database, wherein the user profile is created in real time;
mapping, at the data curation system, the user DNA variance from the user profile with the DNA variances, wherein the mapping is done to identify one or more characteristics associated with the user using one or more machine learning algorithms, wherein the mapping is done in real time; and
generating, at the data curation system, a medical report based on the one or more characteristics, wherein the medical report comprises a plurality of results to be displayed on one or more communication devices.

12. The computer system as recited in claim 11, wherein the user genetic data comprises the user DNA sequences and the genome sequences of the user, wherein the user genetic data is received from one or more input devices in real time.

13. The computer system as recited in claim 11, wherein the one or more machine learning algorithms includes a decision tree algorithm, a random forest algorithm, prediction algorithms, deep learning algorithms and natural language processing algorithm.

14. The computer system as recited in claim 11, wherein the user data comprises name, age, gender, blood group, present disease and disease history of the user, wherein the user data is entered by the user or an operator using the one or more communication devices, wherein the user data is received in real time.

15. The computer system as recited in claim 11, wherein the one or more characteristics comprises the genetic data, observed variant, the genetic variance and diseases related to the genetic variance.

16. The computer system as recited in claim 11, further comprising applying, at the data curation system with the processor, a training dataset on the text data, wherein the training dataset is associated with a predetermined DNA variance data, wherein the training dataset is applied for training the machine to identify genetic data related to DNA variances from the text data.

17. The computer system as recited in claim 11, wherein the genetic data comprises DNA sequences, gene fusion, unique samples of genes, genetic mutation, mutation distribution, genes data, tissue distribution protein-protein interactions, open chromatin data, synthetic lethality data and tissue distribution.

18. The computer system as recited in claim 11, further comprising receiving, at the data curation system with the processor, the user genetic data of a user from one or more input devices and the user data of the user from one or more communication devices.

19. The computer system as recited in claim 11, wherein the plurality of results comprises name, age, gender, blood group, variance explanation, suggestions, user DNA sequence, medical advice, user DNA variances, disease cause and health risk advice.

20. A non-transitory computer-readable storage medium encoding computer executable instructions that, when executed by at least one processor, performs a method for automating curation of genetic data, the method comprising:
extracting, at a computing device, text data from the medical data, wherein medical data is received from a corpus of medical database, wherein the extraction of the text data is performed by using one or more machine learning algorithm, wherein the medical data is received in a plurality of input forms, wherein the corpus of medical database is created from one or more medical databases;
creating, at the computing device, word embedding of words present in the text data in a low dimensional vector space, wherein the word embedding of words is created using one or more methods, wherein the word embedding of words extracts text from the medical data present in the corpus of medical database;
applying, at the computing device, a training dataset on the text data, wherein the training dataset is associated with a predetermined DNA variance data, wherein the training dataset is applied for training a machine to identify genetic data related to DNA variances from the text data, the training dataset is applied in order to train the data curation system to perform automatic curation of the medical data;
identifying, at the computing device, variance explanation from the text data related to the DNA variances, wherein the DNA variances and the variance explanation are identified by analysis of the text data using the one or more machine learning algorithm, wherein the identification is done after applying training dataset on the word embedding of the text data, wherein the identification is done in real time;
creating, at the computing device, a user profile based on user genetic data and user data, wherein the user profile is stored in profile database, wherein the user profile is created in real time;
mapping, at the computing device, the user DNA variance from the user profile with the DNA variances, wherein the mapping is done to identify one or more characteristics associated with the user using one or more machine learning algorithms, wherein the mapping is done in real time; and
generating, at the computing device, a medical report based on the one or more characteristics, wherein the medical report comprises a plurality of results to be displayed on one or more communication devices.
